(19) United States
(12) Patent Application Publication
(10) Pub. No.: US 2013/0267915 A1
(43) Pub. Date: Oct. 10, 2013

(54) MEDICAL GRADE CLEANSING AND NUMBING AGENT COMBINATION PAD

(71) Applicant: David Hoffman, Englewood, CO (US)

(72) Inventor: David Hoffman, Englewood, CO (US)

(21) Appl. No.: 13/856,766

(22) Filed: Apr. 4, 2013

Related U.S. Application Data
(60) Provisional application No. 61/621,771, filed on Apr. 9, 2012.

(51) Int. Cl. A61M 19/00

(52) U.S. Cl. CPC A61M 19/00
USPC 604/290; 604/310

(57) ABSTRACT

A medical grade cleansing and numbing agent combination pad is disclosed. An example combination pad includes a pad impregnated with a cleansing agent and a numbing agent each in separate layers. Both the cleansing agent and the numbing agent are released through a same side of the pad without a user having to turn the pad over to apply both the cleansing agent and the numbing agent to a treatment area at substantially the same time. A barrier prevents the numbing agent from contacting the user during application to the treatment area.

### PRIORITY CLAIM

This application claims the priority benefit of U.S. Provisional Patent Application No. 61/621,771 titled “Cleansing and numbing agent combination pad” of David Hoffman filed on Apr. 9, 2012, hereby incorporated by reference for all that is disclosed as though fully set forth herein.

### BACKGROUND

Antiseptic wipes and pads are well known in the medical field, typically in the form of alcohol prep pads which are used for cleansing an area of the patient's skin prior to applying a bandage or administering a shot.

### BRIEF DESCRIPTION OF THE DRAWINGS

FIG. 1 is perspective view of an example medical grade cleansing and numbing agent combination pad.

FIGS. 2a-b are side views showing different layers of example combination pads.

FIG. 3 is a side view illustrating fissures formed in an example combination pad.

FIGS. 4a-c are side views illustrating removal of a separation material of an example combination pad.

### DETAILED DESCRIPTION

Using a single pad having both antiseptic and anesthetic on separate sides for cleaning open wounds would cause the person using the pad to get anesthetic on their own fingers during application of the antiseptic to the open wound, and then to get blood or other contaminants on their fingers when the pad is turned over to apply the anesthetic to the open wound. Getting an anesthetic on the fingers of the person administering the pad is undesirable, and may even be dangerous (e.g., if the person is unable to properly feel their fingers during subsequent treatment or other procedures). While the person administering the pad may wear gloves, this seems like overkill in some common situations where such a pad may be used (e.g., to clean minor wounds or before administering a shot or IV).

A cleansing and numbing agent combination pad is disclosed herein which alleviates these concerns. An example combination pad may be used for medical purposes (e.g., treating open wounds or as preparation for giving a shot or IV), and includes a single pack prep pad with a cleansing agent in combination with a topical numbing agent or anesthetic. The single pad may be applied topically and performs the functions of cleansing and decontaminating an affected area, while simultaneously or substantially simultaneously de-sensitizing or numbing (at least partially) the affected area in preparation for further medical treatment (e.g., applying a bandage, stitches, or administering a shot or IV). A barrier is used to prevent the numbing agent from contacting the fingers of the person administering the pad (e.g., a healthcare professional).

An example combination pad includes at least two layers, a first layer for the cleansing agent and a second layer for the numbing agent. The two layers can be separated by a separation material which is sufficient to maintain separation between the cleansing agent impregnated in the first layer, and the numbing agent impregnated in the second layer, prior to use. Separation avoids cross-contamination of the cleansing agent and numbing agent, e.g., so that the numbing agent is not weakened or inactivated by mixing with the cleansing agent prior to use. In an example, the separation material may be a sheet that is entirely removed (or at least partially removed) so that the numbing agent can pass through to the first layer for application substantially simultaneously with the cleansing agent.

In another example, the separation material may have a structure or size selected such that mild pressure (e.g., gentle pressure such as would be applied during normal use to clean a wound or prep the skin surface for a shot or IV) is sufficient to cause the numbing agent to pass the separation material during cleansing of the treatment area, allowing the numbing agent to be applied to the skin through the same side of the pad, and at substantially the same time or a short time after, the cleansing agent is applied. In an example, the numbing agent may be a gel which does not readily permeate the first layer prior to use.

Before continuing, it is noted that as used herein, the terms “includes” and “including” mean, but are not limited to, “includes” or “including” and “includes at least” or “including at least.” The term “based on” means “based on” and “based at least in part on.”

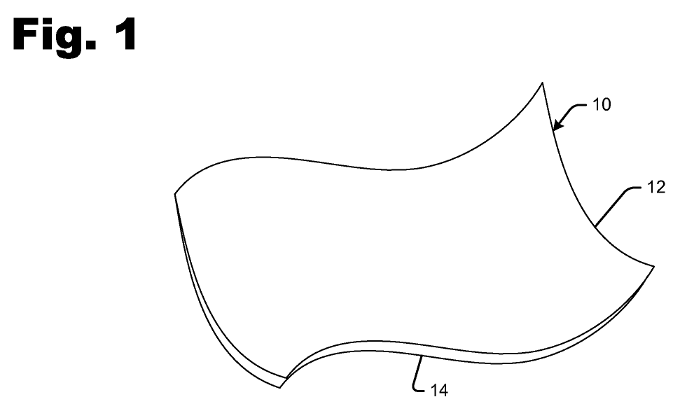

FIG. 1 is a perspective view of an example medical grade cleansing and numbing agent combination pad 10. The medical grade cleansing and numbing agent combination pad 10 (or simply “combination pad”) may be implemented as a single pad including a cleansing agent (e.g., a so-called alcohol “prep pad”) in combination with a numbing agent. In an example, the combination pad 10 may be a gel pad, or at least include one gel layer.

The cleansing agent may be any suitable medical grade cleansing agent for cleaning wounds (e.g., alcohol or other antiseptic). The numbing agent may be any suitable medical grade numbing agent. In an example, the numbing agent is a topical solution and may include, but is not limited to benzocaine, zylocaine, and lydocaine, or other “-caine” numbing agent. It is noted that a higher strength numbing agent may be used for trauma care, while a lower strength numbing agent may be used for administrating routine shots or for home use to cleanse open wounds. The type, strength, and/or concentration of numbing agent may also vary for different types of patients. For example, dosing may be based at least in part on age, sex, and weight of the patient.

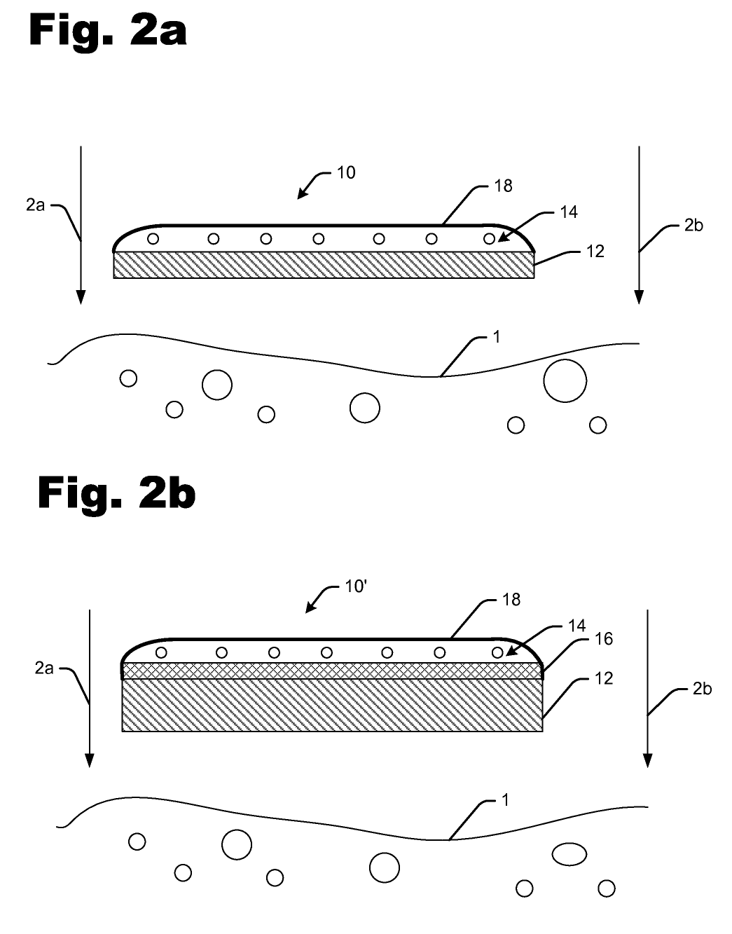

In an example, the pad includes at least two layers. A first layer 12 may be provided for the cleansing agent, and a second layer 14 may be provided for the numbing agent, as can be better seen in FIG. 2.

FIGS. 2a-b are side views showing different layers of example combination pads 10 an 10′. It is noted that FIGS. 2a-b show the combination pad 10 exaggerated in size for purposes of illustration.

In FIG. 2a, the example combination pad 10 includes two layers. A first layer 12 may be the prep pad retaining the cleansing agent. A second layer 14 may be another pad or a gel layer (e.g., without a pad) retained between the top boundary of the first layer 12 and enclosed by a barrier 18. The barrier 18 may be sealed around the perimeter (e.g., onto the edge of the first layer 12) to prevent the numbing agent from leaking out at the sides. The second layer 14 retains the numbing agent. It is noted, however, that any number of layers may be used. Likewise, the function of the different layers may be provided by a single “layer” material but the material having different properties and thus forming “effective” layers within the pad 10. The barrier 18 may be a thicker sheet and/or made of a material that blocks the numbing agent even when the numbing agent is being pressed out through the first layer 12.

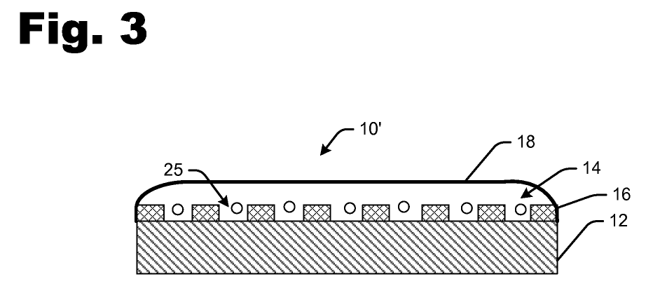

FIG. 2b shows an example combination pad 10′ wherein the first layer 12 and the second layer 14 are separated by a separation material 16. The separation material 16 is configured in such a manner as to provide or maintain additional separation between the cleansing agent and numbing agent prior to use. The separation material 16 may be manufactured of a material and/or thickness such that mild pressure (e.g., the amount of pressure applied to wipe the area during typical use to clean a wound) forces the numbing agent through the separation material 16, as illustrated by fissures 25 illustrated in FIG. 3. Again, the combination pad 10′ is shown exaggerated in size for purposes of illustration.

Fissures may already be present in the separation material 16 (e.g., as in a sponge-like material). In another example, the separation material 16 may be fractured or broken to form cracks or fissures in the separation material 16. Accordingly, the numbing agent can be maintained separate from the cleansing agent, but applied onto the skin at substantially the same time (or a short time after) the cleansing agent has been applied.

It is noted that care should be taken to prevent the barrier 18 from leaking. In an example, the separation material 16 and the barrier 18 are sheets that lay substantially fully over the first and second layers. Accordingly, when stacked on top of one another, the separation material 16 nearly or completely covers the first layer, and the barrier 18 nearly or completely covers the second layer. In an example, the separation material 16 may be an ultra-thin plastic sheet or a sponge-like material.

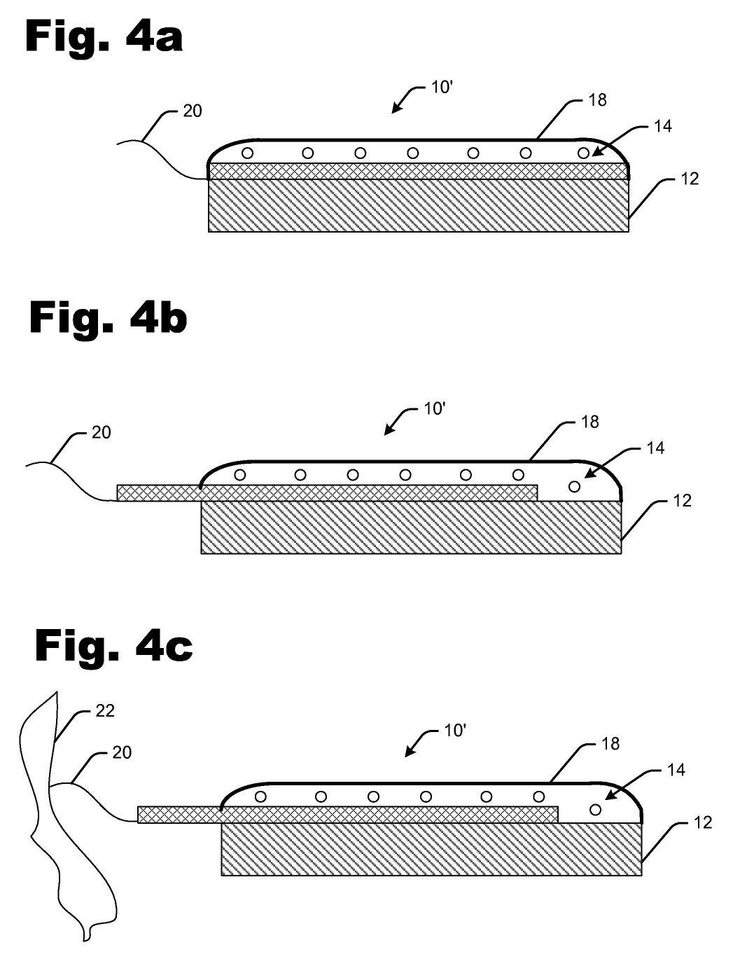

In another example, the separation material 16 may be removed from between the first and second layers. FIGS. 4a-c illustrate the separation material 16 being removed from between the first layer 12 and the second layer 14 by pulling a strip 20. Again, it is noted that the combination pad 10 is shown exaggerated in size for purposes of illustration.

Pulling the strip may be manually by the user as illustrated in FIG. 4b. Pulling the strip may also be automatically (or semi-automatically), by the user unwrapping the combination pad 10 from the product packaging 22, as illustrated in FIG. 4c. That is, as the pad is unwrapped from the product packaging, the strip may be connected to the wrapper and hence remain with the wrapper when the combination pad 10 is pulled away from the wrapper. Removing the wrapper thus removes the separation material 16 so that the numbing agent that was retained by the separation material 16 penetrates in the same direction through the same side of the pad as the cleansing agent (e.g., toward the affected area of skin 1 as illustrated by arrows 2a-b shown in FIG. 2).

Any of these examples enable the numbing agent to be applied to the affected area during use without the user having to turn the combination pad 10 over during use, and without the user's fingers coming into contact with the numbing agent. In this regard, the combination pad 10 may also include a barrier 18. Barrier 18 may be plastic or plastic-coated (or otherwise made impenetrable to the numbing agent during use). Barrier 18 may be provided between the second layer 14 and the user's hand to help prevent the user from getting numbing agent on the user's fingers during use.

The barrier 18 may also serve as a visual indication to the user, which helps the user identify which side to touch (e.g., the plastic coating indicates a handling side), and which side to apply to the affected area (e.g., the other side is an application side). Barrier 18 thus helps the user properly apply the cleansing agent and the numbing agent without getting numbing agent on the user's fingers during use.

Before continuing, it should be noted that the examples described above are provided for purposes of illustration, and are not intended to be limiting. Other devices and/or device configurations may be utilized to carry out the operations described herein.

By way of example, the combination pad 10 is not limited to any particular type or number of layers, and indeed, may be a single layer as mentioned above. For example a single stratified material may be used to provide the different functions as discussed above.

By way of further example, the combination pad 10 may also be configured in any of a wide variety of sizes, types, and/or shapes. For example, the combination pad 10 may be customized for particular uses. Alternatively, the combination pad 10 may be configured for different groups or profiles of users (e.g., minor first aid in the home, EMT, clinical, and hospital uses).

The combination pad may be non-invasive, hypo-allergenic, provides for rapid dispersement, and reduces the occurrence of infection. Examples of the combination pad 10 are disposable and for single-use applications. In another example, the combination pad 10 may include color-coding or some other form of marking or indicia to indicate for a user which side of the combination pad 10 is the application side, use characteristics (e.g., children versus adult anesthetic dosing), and/or to convey other information to the user.

During use, the combination pad 10 is applied topically and performs the functions of cleansing and decontaminating the affected area to reduce or prevent infection, while simultaneously (or substantially simultaneously) de-sensitizing or numbing the affected area.

In an example, the combination pad 10 may be used in advance or in preparation for medical procedures, such as giving a shot, IV, pre-surgical procedure, surgical procedure, post-surgical procedure. The combination pad 10 may also be used in the field by EMTs, e.g., for trauma care.

Users may include, but are not limited to, veterinarians, pediatricians, hospitals, doctor offices, clinics, trauma centers, ambulance EMTs, military, elderly, government, to name only a few examples. Other users are also contemplated.

Although combination pad 10 has been described herein with reference to the figures as it may be used in various environments, by different users, and for different purposes, it is noted that the combination pad 10 is not limited to any one of these in particular. The combination pad 10 may also have other application, as will be readily apparent to those having skill in the art after becoming familiar with the teachings herein.

It is noted that the examples shown and described are provided for purposes of illustration and are not intended to be limiting. Still other embodiments are also contemplated.

1. A medical grade cleansing and numbing agent combination pad, comprising:
a pad impregnated with a cleansing agent and a numbing agent each in separate layers, wherein both the cleansing agent and the numbing agent are released through a same side of the pad without a user having to turn the pad over to apply both the cleansing agent and the numbing agent to a treatment area at substantially the same time; and
a barrier to prevent the numbing agent from contacting the user during application to the treatment area.

2. The cleansing and numbing agent combination pad of claim 1, wherein the pad has at least a first layer impregnated with the cleansing agent in a stacked arrangement with a second layer including the numbing agent as a gel.

3. The cleansing and numbing agent combination pad of claim 1, further comprising a separation sheet between the separate layers, the separation sheet maintaining the cleansing agent apart from the numbing agent.

4. The cleansing and numbing agent combination pad of claim 1, wherein the numbing agent penetrates through fissures separating the first layer during cleansing of the treatment area.

5. The cleansing and numbing agent combination pad of claim 1, further comprising an attachment connected to a separation sheet to remove the separation sheet from between the numbing agent and the cleansing agent by pulling the attachment.

6. The cleansing and numbing agent combination pad of claim 1, further comprising an attachment connected to a separation sheet and a packaging wrapper, wherein opening the packaging wrapper automatically removes the separation sheet from between the numbing agent and the cleansing agent.

7. The cleansing and numbing agent combination pad of claim 1, wherein the pad is a single pack alcohol prep pad with separate numbing agent.

8. The cleansing and numbing agent combination pad of claim 7, wherein the single pack alcohol prep pad cleanses the treatment area while substantially simultaneously de-sensitizing the treatment area in preparation for further medical treatment.

9. The cleansing and numbing agent combination pad of claim 1, wherein the barrier is a plastic sheet.

10. The cleansing and numbing agent combination pad of claim 1, wherein the barrier is a visual indicator to a user of an application side and a handling side.

11. A single pack alcohol-based prep pad with anesthetic, comprising:
means for providing an alcohol-based cleansing agent in a first layer;
means for providing an anesthetic in a second layer; and
wherein the first and second layers release the alcohol-based cleansing agent and the anesthetic of the pad substantially simultaneously during application to cleanse and de-sensitize an affected area without the anesthetic contacting a user's fingers.

12. The prep pad of claim 11, further comprising means for separating the alcohol-based cleansing agent from the anesthetic in the same prep pad prior to application.

13. The prep pad of claim 12, further comprising means for automatically removing the means for separating to combine the anesthetic and the alcohol-based cleansing agent.

14. The prep pad of claim 11, further comprising means for blocking the anesthetic from contacting a user's fingers during application.

15. The prep pad of claim 14, wherein the means for blocking further indicates an application side and a handling side to a user.

16. A method comprising:
releasing a medical grade cleansing agent from a first layer of a prep pad;
releasing a medical grade numbing agent from a second layer of the prep pad at substantially the same time as the medical grade cleansing agent is being released, without a user having to turn over the prep pad to apply both the medical grade numbing agent and the medical grade cleansing agent to a treatment area from the same side of the prep pad, and without the medical grade numbing agent contacting the user.

17. The method of claim 16, further comprising separating the medical grade cleansing agent in the first layer from the medical grade numbing agent in the second layer prior to application.

18. The method of claim 16, further comprising automatically removing a separation sheet from the prep pad when the prep pad is unpackaged.

19. The method of claim 16, further comprising blocking the medical grade numbing agent from penetrating the prep pad on a side in contact with the user's fingers.

20. The method of claim 16, further comprising indicating an application side and a handling side of the prep pad to the user by a barrier on the prep pad.
